(19) United States
(12) Patent Application Publication
(10) Pub. No.: US 2023/0158177 A1
(43) Pub. Date: May 25, 2023

(54) HYPOXIA-TARGETING CONTRAST AGENT AND METHODS OF USE THEREOF

(71) Applicants: Vikram Kodibagkar, Tempe, AZ (US); Babak Moghadas, Baltimore, MD (US)

(72) Inventors: Vikram Kodibagkar, Tempe, AZ (US); Babak Moghadas, Baltimore, MD (US)

(73) Assignee: Arizona Board of Regents on behalf of Arizona State University, Scottsdale, AZ (US)

(21) Appl. No.: 18/056,349

(22) Filed: Nov. 17, 2022

Related U.S. Application Data
(60) Provisional application No. 63/281,358, filed on Nov. 19, 2021.

(51) Int. Cl. A61K 49/10; C07D 233/91; C07F 15/02

(52) U.S. Cl. CPC A61K 49/106; C07D 233/91; C07F 15/025

(57) ABSTRACT

The present disclosure provides for a novel hypoxia-targeting contrast agent for noninvasive detection of hypoxia using magnetic resonance imaging. Also disclosed is a process of preparing the novel hypoxia-targeting contrast agent, as well as methods of using the novel hypoxia-targeting contrast agent.

### CROSS-REFERENCE TO RELATED APPLICATIONS

This application claims priority to and the benefit of U.S. Provisional Application No. 63/281,358, filed Nov. 19, 2021, the disclosure of which is incorporated herein by reference in its entirety.

### BACKGROUND OF THE INVENTION

Hypoxia, defined as lack of oxygen, has many known ramifications on the outcome of therapy in any condition. Hence, it is very important to study hypoxia. The gold standard method to detect hypoxia, IHC staining of pimonidazole, is invasive. However, there is much interest in developing new and noninvasive methods to investigate hypoxia in different tissues.

Magnetic Resonance Imaging (MRI), a non-invasive imaging modality provides high resolution, does not need radioactive molecules, can provide information from deep tissue, and is sensitive to different soft tissues. It is widely used in anatomical and functional imaging of the body. The contrast in MRI is generated based on the intrinsic properties of the tissue (such as T₁, T₂, p). MRI contrast can be enhanced by perfusing the tissue with a contrast agent that can modify these properties. Contrast agents have attracted a lot of attention because they can be modified with targeting groups to shed light on some physiological and biological questions such as presence of hypoxia in a tissue.

Gadolinium (Gd), which is a strongly paramagnetic heavy metal, is routinely and widely used as an MRI contrast agent by chelation with a biocompatible ligand, which is typically cleared through the kidneys. While widely used, there are serious concerns for patients with impaired kidney function as well as recent studies showed Gd accumulation in the bone and brain. Iron as a physiological ion is also capable of generating contrast in MRI images with the added advantage of resorption into the tissue.

Thus, there is a need in the art for compounds with no or low toxocity that are effective in detecting hypoxia as well as methods of use thereof as contract agent for detecting, monitoring, and diagnosing hypoxia. The present invention satisfies this unmet need.

### SUMMARY OF THE INVENTION

The present invention provides a compound having the structure of Formula (I):

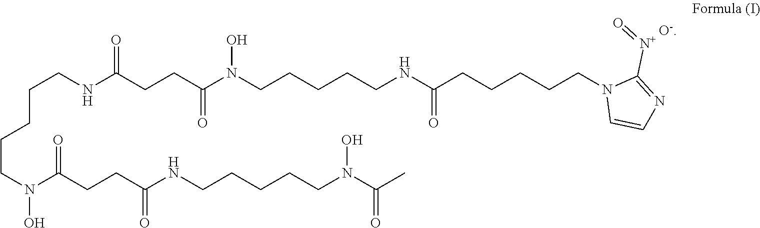

In another aspect, the present invention provides a metal chelate comprising a compound of Formula (I) and a metal.

In some embodiments, the metal is selected from iron counterion, copper counterion, cobalt counterion, manganese, nickel, zinc, zirconium, gallium, neodymium, europium, gadolinium, terbium, dysprosium, holmium, erbium, thulium, ytterbium, lutetium, or any combination thereof.

In one embodiment, the metal is iron.

In some embodiments, the iron has an oxidation state of +2 or +3. In one embodiment, the iron has an oxidation state of +3.

In another aspect, the present invention provides a process for the preparation of the metal chelate of the present invention. In some embodiment, the process comprises contacting a compound of Formula (I) with a metal complex to form the metal chelate.

In one embodiment, the metal complex is FeCl₃.

In some embodiments, the process further comprises contacting the compound

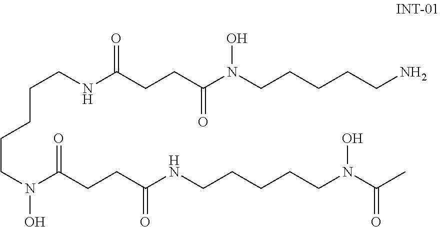 [SMILES: CC(=O)N(O)CCCCCNC(=O)CCC(=O)N(O)CCCCCNC(=O)CCC(=O)N(O)CCCCCN]

with the compound

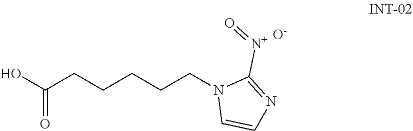

in the presence of a coupling reagent, an amine base, 1-hydroxybenzotriazole monohydrate (HOBt.xH₂O), and a solvent to obtain the compound of Formula (I).

In some embodiments, the coupling reagent is selected from 3-[bis(dimethylamino)methyliumyl]-3H-benzotriazol-1-oxide hexafluorophosphate (HBTU), benzotriazol-1-yloxytripyrrolidinophosphonium hexafluorophosphate (PyBOP), 1-ethyl-3-(3-dimethylaminopropyl)carbodiimide (EDC), N-hydroxysuccinimide (NHS), or any combination thereof. In one embodiment, the coupling reagent is 3-[bis(dimethylamino)methyliumyl]-3H-benzotriazol-1-oxide hexafluorophosphate (HBTU).

In one embodiment, the amine base is N,N-diisopropylethylamine.

In one embodiment, the solvent is dimethylformamide.

In some embodiments, the process further comprises contacting the compound

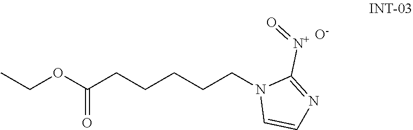

with an acid and a solvent to form the compound (6-(2-nitro-1H-imidazol-1-yl)hexanoic acid) (INT-02).

In some embodiments, the acid is in a concentration of about 12 M.

In one embodiment, the acid is HCl.

In some embodiments, the solvent is selected from water, dimethylformamide (DMF), methanol, dimethyl sulfoxide (DMSO), or any combination thereof. In one embodiment, the solvent is water. In one embodiment, the solvent is dimethylformamide. In one embodiment, the solvent is water and dimethylformamide.

In some embodiments, the process further comprises contacting the compound

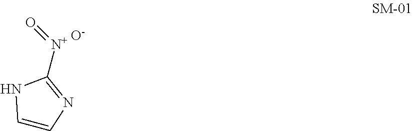

with the compound

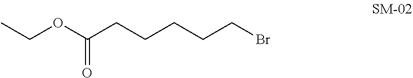

in the presence of base and a solvent to form ethyl-(-2-nitroimidazoyl)hexanoate (compound INT-03).

In one embodiment, the base is K₂CO₃. In another embodiment, the base is Na₂CO₃.

In one embodiment, the solvent is acetonitrile.

In one aspect, the present invention provides a method of imaging hypoxic tissue regions. In some embodiments, the method comprises applying the metal chelate of the present invention to the tissue, and using magnetic resonance imaging (MRI) to detect the metal chelate.

In another aspect, the present invention provides a method of perfusion MRI. In some embodiments, the method comprises applying the metal chelate of the present invention to the tissue, and using MRI to detect the metal chelate in a region of the tissue.

In some embodiments, the region of the tissue is hypoxic.

In some embodiments, the metal chelate is dosed at a concentration up to about 400 μM. In some embodiments, the metal chelate is dosed at a concentration between about 50 μM and about 400 μM.

In some embodiments, the metal chelate is dosed at a concentration between about 0.05 mmol/kg body wt to about 1.0 mmol/kg body wt. In one embodiment, the metal chelate is dosed at a concentration of about 0.2 mmol/kg body wt.

In various embodiments, the metal chelate comprises any metal described herein. For example, in some embodiments, the metal of the metal chelate is selected from iron, copper, cobalt, manganese, nickel, zinc, zirconium, gallium, neodymium, europium, gadolinium, terbium, dysprosium, holmium, erbium, thulium, ytterbium, lutetium, or any combination thereof. In one embodiment, the metal of the metal chelate is iron. In some embodiments, the iron has an oxidation state of +2 or +3. In one embodiment, the iron has an oxidation state of +3.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts a representative ¹H NMR spectrum of Deferoxamine B (INT-01 or DFOB) in DMSO-d6, ¹H NMR (550 MHz, DMSO-d6): δ 7.8 (211, amide), 3.46 (6H, —CH₂—(N—OH)—), 3 (4H, —CH₂—(NH)—).

FIG. 2 depicts a representative ¹H NMR spectrum of Formula (I) in DMSO-d6 after column chromatography, ¹H NMR (550 MHz, DMSO-d6): δ 7.8 (2H, amide), 3.46 (6H, —CH₂—(N—OH)—), 3 (6H, —CH₂—(NH)—).

FIG. 3 depicts representative results of ESI-MS of INT-01. 50/50 A/B, A: 20 mM ammonium formate in water, and B: 20 mM ammonium formate in MeOH.

FIG. 4, comprising FIG. 4A and FIG. 4B, depicts representative results of ESI-MS of Formula (I) and metal chelate after dialysis. 50/50 A/B, A: 20 mM ammonium formate in water, and B: 20 mM ammonium formate in MeOH. FIG. 4A depicts representative results of ESI-MS of Formula (I) after dialysis. 50/50 A/B, A: 20 mM ammonium formate in water, and B: 20 mM ammonium formate in MeOH. FIG. 4B depicts representative results of ESI-MS of metal chelate after dialysis. 50/50 A/B, A: 20 mM ammonium formate in water, and B: 20 mM ammonium formate in MeOH.

FIG. 5, comprising FIG. 5A and FIG. 5B, depicts representative results demonstrating the fibroblast 3T3 cell viability assay using Alamar blue after being exposed to Feroxamine B conjugate of 2-nitroimidazole (FOBNI) over different periods in normoxic condition represents the data using the control for 4 h for all the time points and represents the data using the control in each time point the * in each case shows the statistical significance compared to control. FIG. 5A depicts representative results demonstrating the fibroblast 3T3 cell viability assay using Alamar blue after being exposed to FOBNI over different periods in normoxic condition represents the data using the control for 4 h for all the time points. FIG. 5B depicts representative results demonstrating the fibroblast 3T3 cell viability assay using Alamar blue after being exposed to FOBNI over different periods in normoxic condition represents the data using the control in each time point the * in each case shows the statistical significance compared to control.

FIG. 6, comprising FIG. 6A and FIG. 6B, depicts representative results demonstrating the fibroblast 3T3 cell viability assay using Alamar blue after being exposed to FOBNI over different periods in hypoxic condition represents the data using the control for 4 h for all the time points and represents the data using the control in each time point the * in each case shows the statistical significance compared to control. FIG. 6A depicts representative results demonstrating the fibroblast 3T3 cell viability assay using Alamar blue after being exposed to FOBNI over different periods in hypoxic condition represents the data using the control for 4 h for all the time points. FIG. 6B depicts representative results demonstrating the fibroblast 3T3 cell viability assay using Alamar blue after being exposed to FOBNI over different periods in hypoxic condition represents the data using the control in each time point the * in each case shows the statistical significance compared to control.

FIG. 7, comprising FIG. 7A and FIG. 7B, depicts representative T₁ and T₂ maps in ms and concentration maps in mM Fe for Feroxamine B (FOB) and FOBNI. FIG. 7A depicts representative T₁ and T₂ maps in ms and concentration maps in mM Fe for FOB. FIG. 7B depicts representative T₁ and T₂ maps in ms and concentration maps in mM Fe for FOBNI.

FIG. 8, comprising FIG. 8A and FIG. 8B, depicts representative results of relaxivity study of FOB and FOBNI, respectively. FIG. 8A depicts representative results of the relaxivity study of FOB with the fit curves representing the data as (R₁=2.06±0. 06)[C]+(0.49±0.04) (in red) and R₂=(2.75±0.06)[C]+(3.75±0.08) (in gray). FIG. 8B depicts representative results of the relaxivity study of FOBNI with the linear fit equations as R₁=(0.51±0.1)[C]+(0.44±0.03) (in red) and R₂=(4.51±0.14) [C]+(3.87±0.19) (in gray).

FIG. 9, comprising FIG. 9A through FIG. 9F, depicts representative T₁ and T₂ maps of cell pellets exposed to FOB, exposed to FOBNI, and a control. FIG. 9A depicts representative T₁ map of control cell pellets. FIG. 9B depicts representative T₂ map of control cell pellets. FIG. 9C depicts representative T₁ map of cell pellets exposed to FOB. FIG. 9D depicts representative T₂ map of cell pellets exposed to FOB. FIG. 9E depicts representative T₁ map of cell pellets exposed to FOBNI. FIG. 9F depicts representative T₂ map of cell pellets exposed to FOBNI.

### DETAILED DESCRIPTION

The present invention is based, in part, on the unexpected results that a metal chelate comprising a compound having the structure:

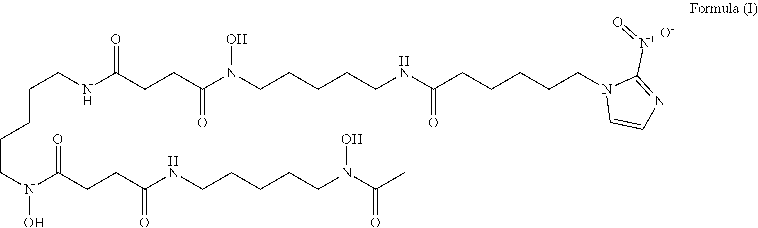

and an iron was non-toxic and an effective contrast agent for imaging hypoxic tissue regions. Thus, in one aspect, the present invention provides a compound of Formula (I). In another aspect, the present invention provides a metal chelate comprising a compound of Formula (I) and a metal. In one embodiment, the metal chelate is a contrast agent. In one embodiment, the metal chelate is an magnetic resonance imaging (MRI) contrast agent. In one aspect, the present invention relates to a process of preparing the metal chelate of the present invention.

In one aspect, the present invention also relates to a method of imaging hypoxic tissue regions, the method comprising applying the metal chelate of the present disclosure to the tissue and using MRI to detect the metal chelate. In another aspect, the present invention provides a method of perfusion MRI, the method comprising applying the metal chelate of the present invention to the tissue and using MRI to detect the metal chelate in a region of the tissue. In some embodiments, the region of the tissue is hypoxic.

In another aspect, the present invention provides a method of detecting, monitoring, and diagnosing hypoxia, the method comprising applying the metal chelate of the present invention to a tissue, detecting the metal chelate in a region of the tissue using MRI, and determining that the tissue is hypoxic when the metal chelate is detected.

### Definitions

Before the present compositions and methods are described, it is to be understood that this invention is not limited to the particular processes, compositions, or methodologies described, as these may vary. It is also to be understood that the terminology used in the description is for the purpose of describing the particular versions or embodiments only and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

As used herein, the terms below have the meanings indicated.

It must also be noted that as used herein and in the appended claims, the singular forms “a,” “an,” and “the” include plural reference unless the context clearly dictates otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. For example, about 50 mm means in the range of 45 mm to 55 mm.

The transitional term “comprising,” which is synonymous with “including,” “containing,” or “characterized by,” is inclusive or open-ended and does not exclude additional, unrecited elements or method steps.

In embodiments or claims where the term “comprising” is used as the transition phrase, such embodiments can also be envisioned with replacement of the term “comprising” with the terms “consisting of” or “consisting essentially of.”

As used herein, the term “consists of” or “consisting of” means that the composition or the method includes only the elements, steps, or ingredients specifically recited in the particular claimed embodiment or claim.

As used herein, the term “consisting essentially of” or “consists essentially of” means that the composition or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the composition or method that treats the specified condition (e.g., nutrient depletion) is the specifically recited therapeutic(s) in the particular embodiment or claim.

As used herein, the term “DFOBNI” may be used interchangeably with “Deferoxamine B conjugate of 2-nitroimidazole,” “Formula (I),” and “a compound of Formula (I).”

As used herein, the term “DFOB” may be used interchangeably with “Deferoxamine B,” “INT-01,” and “a compound of INT-01.”

As used herein, the term “FOBNI” may be used interchangeably with “Feroxamine B conjugate of 2-nitroimidazole,” and “metal chelate.”

As used herein, the term “FOB” may be used interchangeably with “Feroxamine B.”

An example of the structure of FOB with an iron atom as the metal being chelated may be found in Bellotti et al., Molecules 2021, 26, 3255 which is hereby incorporated by reference in its entirety. DFOBNI chelates metals with the its hydroxamic groups in a similar manner to FOB.

Any embodiment provided herein may be combined with any one or more of the other embodiments, unless otherwise stated and provided the combination is not mutually exclusive.

### DETAILED DESCRIPTION

Inadequate supply of oxygen, known as hypoxia, is a tissue microenvironmental feature that can affect the outcome of different pathologies. It is also defined as constrained molecular oxygen electron transport. Hypoxia influences the tissue microenvironment and deviates the tissue and cell function from normal. For instance, it negatively affects the immune system response, tumor invasiveness, and metastasis. There are extensive studies on hypoxia in cancer, traumatic brain injury, and tissue implantation. Hypoxia can be the result of many factors, including impaired oxygen delivery to the tissue because of abnormal angiogenesis, disruption of the blood vessels, or limited oxygen diffusion. For example, in cancer, highly proliferating tumor cells demand more oxygen and nutrients, which results in the formation of new and disorganized blood vessels (neoangiogenesis). These vessels are not fully functional, are leaky, and fail to rectify reduced oxygen levels. Some of the important complications known from and caused by hypoxia are resistance to chemotherapy and radiotherapy, increased tumor aggression, metastasis in cancer, alteration of therapy and neurological outcomes in TBI and success of transplanted tissue or implanted devices; these may directly impact patients' survival. Under hypoxic conditions, cells' energy production pathway alters to glycolysis to reduce their need for oxygen. Due to lack of oxygen, radiation is not very effective in hypoxic tumors.

Considering the importance of hypoxia, it has become an important therapeutic target for monitoring tumor progression, treatment response, and the development of hypoxia-activated prodrugs. Since the hypoxic condition is harsh for normal cells, the number of surviving cells is limited; this requires very precise methods and techniques to detect and study hypoxia. There are two key approaches to studying hypoxia: using endogenous markers like HIF1-alpha, carbonic anhydrase IX (CA IX), and endoplasmic reticulum disulphide oxidase 1α (ERO1α) and or exogenous markers that target hypoxia. The manifestation of the extent of hypoxia in tissue depends on the applied techniques and methods. For example, it is reported that detected hypoxic regions with endogenous markers do not correlate exactly with the result of the pimonidazole staining approach translating; the pimonidazole staining can detect severe hypoxic regions and may not accumulate in the HIF1 active regions.

Nitroimidazoles accumulate in hypoxic tissues and hypoxic cells. The main proposed mechanism for the accumulation of these compounds follows the same reasoning for resistance to radiotherapy: lack of oxygen and oxygen radicals. Initially, the nitroimidazoles undergo a bio-reduction by nitroreductase enzymes and cofactors, such as NADPH; in this process the nitro group acquires an electron and generates a radical in oxygen-available (normoxic) conditions. Molecular oxygen oxidizes the nitro radical to reproduce the parent molecule, and subsequently the superoxide anion is transformed to hydrogen peroxide by superoxide dismutase; however, in the absence of oxygen they create a covalent bound with thiol groups. This important feature has been the main motive in generatinghypoxia-activated prodrugs, imaging agents, and antibiotics for anaerobic organisms. Pimonidazole, the most successful member of the nitroimidazole family, has been used to study hypoxia in animals and humans. While some evidence shows the binding of nitroimidazoles to hypoxic cells and the surrounding oxygen levels are correlated, there is no published report on calibration for retention of these molecules as a function of oxygen tension. Moreover, there is no clear and detailed mechanism of the binding reaction of nitroimidazoles and cellular components.

Even though fluorescence spectroscopy and immunohistochemistry approaches to study hypoxia are very accurate and provide high spatial resolutions, noninvasive investigation of hypoxia with advanced techniques can eliminate the potential complications resulting from invasive procedures compensating the resolution and can provide further vital information about tissue microenvironment and tissue characteristics like pharmacokinetic properties shedding light on personalized therapy. Reported approaches to detect nitroimidazoles include scintigraphy, ¹⁹F-MRI, positron emission tomography (PET), immunohistochemistry, high-frequency ultra-sound microscopy, SPECT, near infrared (NRI), Electron Paramagnetic Resonance (EPR) and proton Magnetic resonance imaging (MRI). Owing to its unique package of features like high resolution, deep tissue penetration, providing anatomical and functional images and more importantly being noninvasive, MR-based techniques to study hypoxia are safe, versatile, and favorable to scientists. The contrast in MR images, which comes from the influence of intrinsic properties of the environment on the magnetic properties of protons, can be tailored for specific purposes. For example, the presence of MR contrast agents alters the image contrast by changing the relaxation rate of the surrounding protons. The relaxation rate of tissue affected by the contrast agent is described by:

Rᵢ=[C]rᵢ+R₀

where [C] is the concentration of the contrast agent, rᵢ is the relaxivity of the contrast agent, and R₀ is the inherent relaxation rate of the tissue. There have been multiple efforts to design novel contrast agents to address different biological and physiological questions. Hypoxia, a therapeutic target, can be mapped with MRIif a hypoxia-targeting contrast agent is introduced to the region of interest. Gadolinium tetraazacyclododecanetetraacetic acid monoamide conjugate of 2-nitroimidazole (GdDO3NI) was developed to target hypoxia and visualize it utilizing MRI. Because GdDO3NI has the same targeting moiety of pimonidazole, it is assumed to follow the same mechanism of reactivity under hypoxic conditions with proteins. The contrast agent has shown successful results in prior studies, in subcutaneous prostate cancer, and in lung tumors. This successful study opens up the opportunity to use GdDO3NI in other pathologies like TBI.

Gd, which is a strongly paramagnetic heavy metal, is routinely and widely used as an MR contrast agent by chelation with a biocompatible ligand which is typically cleared through the kidneys. While widely used, there are serious concerns for patients with impaired kidney function, and recent studies showed Gd accumulation in the bone and brain tissue, even in patients with no renal impairment. Moreover, the reported potential drawbacks of Gd-based contrast agents include cytotoxicity, negative effects on cell proliferation, and low cellular uptake. These are convincing enough for researchers to investigate and develop other non-Gd-based contrast agents to replace the existing contrast agents. Iron, a physiological ion, is capable of generating contrast in MR images due toits paramagnetic properties. Iron is the most abundant metal on earth, and it has important roles in physiological processes in the human body such as oxygen transport, DNA synthesis, and the formation of several biologically important enzymes. Iron-based contrast agents are considered versatile and capable of being engineered for different applications with the highest sensitivity. Deferoxamine B (DFOB) is a linear trihydroxamic acid siderophore. Siderophores are compounds that are secreted in bacteria as a result of Fe (III) deficiency. DFOB and other siderophores are efficient in gleaning Fe (III) from an inorganic or biological source.

### Compounds

In one aspect, the present invention provides a compound of Formula (I):

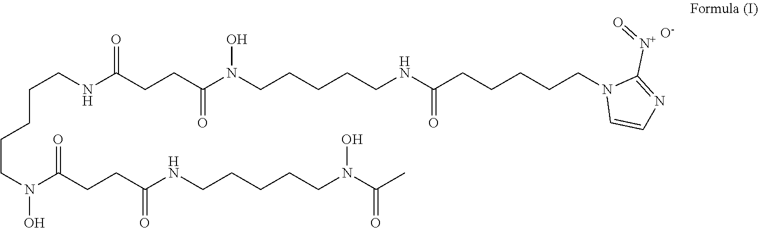

In another aspect, the present invention provides a metal chelate comprising a compound of Formula (I) and a metal.

In some embodiments of the metal chelate, the metal is selected from iron, copper, cobalt, manganese, nickel, zinc, zirconium, gallium, neodymium, europium, gadolinium, terbium, dysprosium, holmium, erbium, thulium, ytterbium, lutetium, or any combination thereof.

In some embodiments of the metal chelate, the metal is iron.

In some embodiments of the metal chelate, the iron has an oxidation state of +2 or +3.

In some embodiments of the metal chelate, the iron has an oxidation state of +2.

In some embodiments of the metal chelate, the iron has an oxidation state of +3.

In some embodiments of the metal chelate, the metal has an oxidation state selected from the group consisting of +1, +2, +3, +4, +5, +6, and +7.

In one embodiment, the metal chelate is a contrast agent. In one embodiment, the metal chelate is an magnetic resonance imaging (MRI) contrast agent.

### Process of Preparation

In one aspect, the present invention relates to a process of preparing the metal chelate of the present invention.

In some embodiments, the process comprises the synthesis of Formula (I) as outlined in Scheme 1.

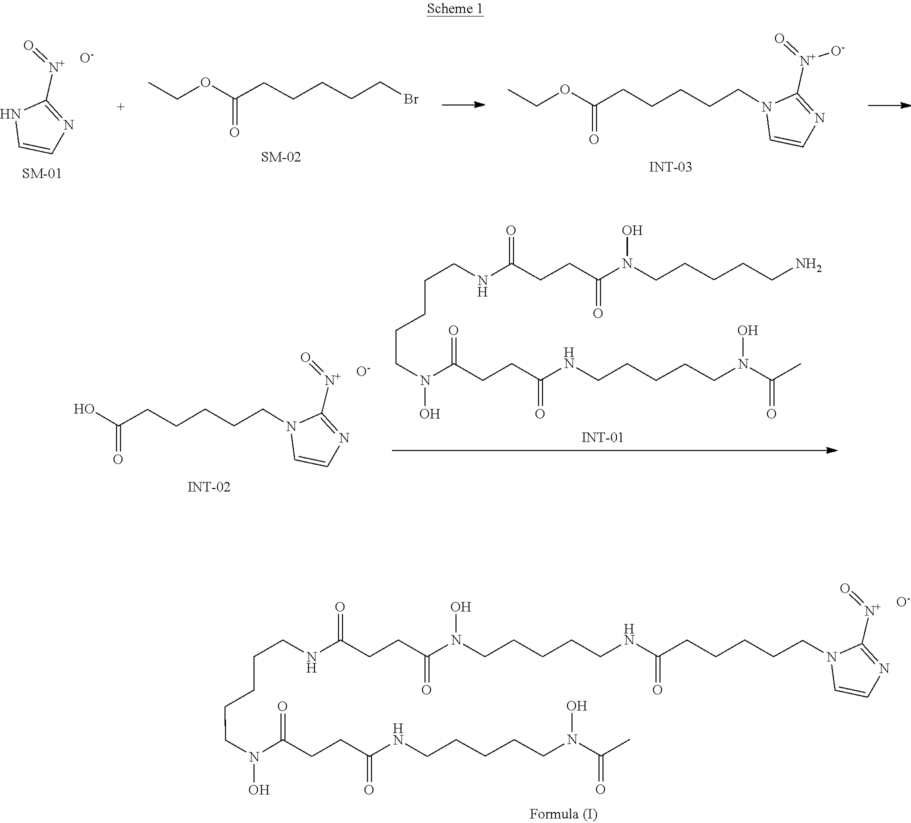

Some embodiments of the process for the preparation of the metal chelate, the process comprises contacting a compound of Formula (I) with a metal complex to form the metal chelate.

In some embodiments of the process for the preparation of the metal chelate, the metal complex is FeCl₃.

In some embodiments of the process for the preparation of the metal chelate, the process further comprises contacting the compound

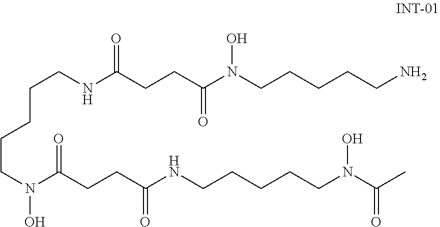 [SMILES: CC(=O)N(O)CCCCCNC(=O)CCC(=O)N(O)CCCCCNC(=O)CCC(=O)N(O)CCCCCN]

with the compound

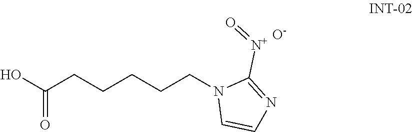

in the presence of a coupling reagent, an amine base, HOBt.xH₂O, and a solvent to obtain the compound of Formula (I).

In some embodiments of the obtaining of the compound of formula (I), the coupling reagent is selected from 3-[bis(dimethylamino)methyliumyl]-3H-benzotriazol-1-oxide hexafluorophosphate (HBTU), benzotriazol-1-yloxytripyrrolidinophosphonium hexafluorophosphate (PyBOP), 1-ethyl-3-(3-dimethylaminopropyl)carbodiimide (EDC), N-hydroxysuccinimide (NHS), or any combination thereof.

In some embodiments of the obtaining of the compound of formula (I), the coupling reagent is 3-[bis(dimethylamino)methyliumyl]-3H-benzotriazol-1-oxide hexafluorophosphate (HBTU).

In some embodiments of the obtaining of the compound of formula (I), the amine base is N,N-diisopropylethylamine (DIPEA).

In some embodiments of the obtaining of the compound of formula (I), the solvent is dimethylformamide (DMF).

In some embodiments of the process for the preparation of the metal chelate, the process further comprises contacting the compound

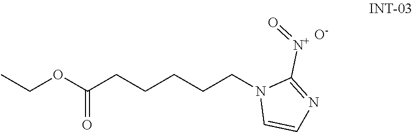

with an acid and a solvent to form the compound INT-02.

In some embodiments of the forming of INT-02, the acid is HCl.

In some embodiments of the forming of INT-02, the solvent is selected from the group consisting of water, dimethylformamide (DMF), methanol, dimethyl sulfoxide (DMSO), or a combination thereof.

In some embodiments of the forming of INT-02, the solvent is water.

In some embodiments of the forming of INT-02, the solvent is dimethylformamide.

In some embodiments of the forming of INT-02, the solvent is water and dimethylformamide.

In some embodiments of the forming of INT-02, the acid is in a concentration of 12 M.

In some embodiments of the process for the preparation of the metal chelate, the process further comprises contacting the compound

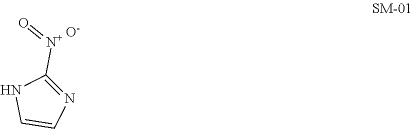

with the compound

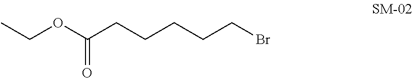

in the in the presence of base and a solvent to form the compound INT-03.

In some embodiments of the forming of INT-03, the base is K₂CO₃.

In some embodiments of the forming of INT-03, the base is Na₂CO₃.

In some embodiments of the forming of INT-03, the solvent is acetonitrile.

### Methods of Use

In one aspect, the present invention relates to a method of imaging hypoxic tissue regions, the method comprising: applying the metal chelate of the present disclosure to the tissue, and using magnetic resonance imaging (MRI) to detect the metal chelate.

In some embodiments of the method of imaging hypoxic tissue regions, the metal of the metal chelate is any metal disclosed herein. For example, in some embodiments of the method of imaging hypoxic tissue regions, the metal of the metal chelate is selected from iron, copper, cobalt, manganese, nickel, zinc, zirconium, gallium, neodymium, europium, gadolinium, terbium, dysprosium, holmium, erbium, thulium, ytterbium, lutetium, or any combination thereof.

In some embodiments of the method of imaging hypoxic tissue regions, the metal of the metal chelate is iron.

In some embodiments of the method of imaging hypoxic tissue regions, the iron has an oxidation state of +2 or +3.

In some embodiments of the method of imaging hypoxic tissue regions, the iron has an oxidation state of +3.

In some embodiments of the method of imaging hypoxic tissue regions, the metal has an oxidation state selected from the group consisting of +1, +2, +3, +4, +5, +6, and +7.

In some embodiments of the method of imaging hypoxic tissue regions, the metal chelate is dosed at a concentration up to about 400 μM.

In some embodiments of the method of imaging hypoxic tissue regions, the metal chelate is dosed at a concentration between about 50 μM and about 400 μM, between about 75 μM and about 400 μM, between about 100 μM and about 400 μM, between about 125 μM and about 400 μM, between about 150 μM and about 400 μM, between about 175 μM and about 400 μM, between about 200 μM and about 400 μM, between about 225 μM and about 400 μM, between about 250 μM and about 400 μM, between about 275 μM and about 400 μM, between about 300 μM and about 400 μM, between about 325 μM and about 400 μM, between about 350 μM and about 400 μM, between about 375 μM and about 400 μM, between about 50 μM and about 375 μM, between about 50 μM and about 350 μM, between about 50 μM and about 325 μM, between about 50 μM and about 300 μM, between about 50 μM and about 275 μM, between about 50 μM and about 250 μM, between about 50 μM and about 225 μM, between about 50 μM and about 200 μM, between about 50 μM and about 175 μM, between about 50 μM and about 150 μM, between about 50 μM and about 125 μM, between about 50 μM and about 100 μM, between about 50 μM and about 75 μM, between about 75 μM and about 375 μM, between about 100 μM and about 350 μM, between about 125 μM and about 325 μM, between about 150 μM and about 300 μM, between about 175 μM and about 275 μM, or between about 200 μM and about 250 μM. In some embodiments of the method of imaging hypoxic tissue regions, the metal chelate is dosed at a concentration of about 50 μM, about 75 μM, about 100 μM, about 125 μM, about 150 μM, about 175 μM, about 200 μM, about 225 μM, about 250 μM, about 275 μM, about 300 μM, about 325 μM, about 350 μM, about 375 μM, about 400 μM, or any range between any two of these values, including endpoints. In an embodiment of the method of imaging hypoxic tissue regions, the metal chelate is dosed at a concentration between about 50 μM and about 400 μM.

In some embodiments of the method of imaging hypoxic tissue regions, the metal chelate is dosed at a concentration between about 0.05 mmol/kg body wt to about 1.0 mmol/kg body wt, about 0.1 mmol/kg body wt to about 1.0 mmol/kg body wt, about 0.2 mmol/kg body wt to about 1.0 mmol/kg body wt, about 0.3 mmol/kg body wt to about 1.0 mmol/kg body wt, about 0.4 mmol/kg body wt to about 1.0 mmol/kg body wt, about 0.5 mmol/kg body wt to about 1.0 mmol/kg body wt, about 0.6 mmol/kg body wt to about 1.0 mmol/kg body wt, about 0.7 mmol/kg body wt to about 1.0 mmol/kg body wt, about 0.8 mmol/kg body wt to about 1.0 mmol/kg body wt, about 0.9 mmol/kg body wt to about 1.0 mmol/kg body wt, about 0.05 mmol/kg body wt to about 0.9 mmol/kg body wt, about 0.05 mmol/kg body wt to about 0.8 mmol/kg body wt, about 0.05 mmol/kg body wt to about 0.7 mmol/kg body wt, about 0.05 mmol/kg body wt to about 0.6 mmol/kg body wt, about 0.05 mmol/kg body wt to about 0.5 mmol/kg body wt, about 0.05 mmol/kg body wt to about 0.4 mmol/kg body wt, about 0.05 mmol/kg body wt to about 0.3 mmol/kg body wt, about 0.05 mmol/kg body wt to about 0.2 mmol/kg body wt, about 0.05 mmol/kg body wt to about 0.1 mmol/kg body wt. about 0.1 mmol/kg body wt to about 0.9 mmol/kg body wt, about 0.2 mmol/kg body wt to about 0.8 mmol/kg body wt, about 0.3 mmol/kg body wt to about 0.7 mmol/kg body wt, or about 0.4 mmol/kg body wt to about 0.6 mmol/kg body wt. In some embodiments of the method of imaging hypoxic tissue regions, the metal chelate is dosed at a concentration of about 0.05 mmol/kg body wt, about 0.1 mmol/kg body wt, about 0.2 mmol/kg body wt, about 0.3 mmol/kg body wt, about 0.4 mmol/kg body wt, about 0.5 mmol/kg body wt, about 0.6 mmol/kg body wt, about 0.7 mmol/kg body wt, about 0.8 mmol/kg body wt, about 0.9 mmol/kg body wt, about 1.0 mmol/kg body wt, or any range between any two of these values, including endpoints. In certain embodiments of the method of imaging hypoxic tissue regions, the metal chelate is dosed at a concentration between about 0.05 mmol/kg body wt to about 1.0 mmol/kg body wt. In an embodiment of the method of imaging hypoxic tissue regions, the metal chelate is dosed at a concentration of about 0.2 mmol/kg body wt.

In one aspect, the present invention also provides a method of perfusion MRI, the method comprising applying the metal chelate of the present invention to the tissue and using MRI to detect the metal chelate in a region of the tissue.

In some embodiments of the method of perfusion MRI, the metal of the metal chelate is any metal disclosed herein. For example, in some embodiments of the method of perfusion MRI, the metal of the metal chelate is selected from iron, copper, cobalt, manganese, nickel, zinc, zirconium, gallium, neodymium, europium, gadolinium, terbium, dysprosium, holmium, erbium, thulium, ytterbium, lutetium, or any combination thereof.

In some embodiments of the method of perfusion MRI, the metal of the metal chelate is iron. In some embodiments of the method of perfusion MRI, the iron has an oxidation state of +2 or +3. In some embodiments of the method of perfusion MRI, the iron has an oxidation state of +3.

In some embodiments of the method of perfusion MRI, the metal chelate is dosed at a concentration up to about 400 μM. In some embodiments of the method of perfusion MRI, the metal chelate is dosed at a concentration between about 50 μM and about 400 μM, between about 75 μM and about 400 μM, between about 100 μM and about 400 μM, between about 125 μM and about 400 μM, between about 150 μM and about 400 μM, between about 175 μM and about 400 μM, between about 200 μM and about 400 μM, between about 225 μM and about 400 μM, between about 250 μM and about 400 μM, between about 275 μM and about 400 μM, between about 300 μM and about 400 μM, between about 325 μM and about 400 μM, between about 350 μM and about 400 μM, between about 375 μM and about 400 μM, between about 50 μM and about 375 μM, between about 50 μM and about 350 μM, between about 50 μM and about 325 μM, between about 50 μM and about 300 μM, between about 50 μM and about 275 μM, between about 50 μM and about 250 μM, between about 50 μM and about 225 μM, between about 50 μM and about 200 μM, between about 50 μM and about 175 μM, between about 50 μM and about 150 μM, between about 50 μM and about 125 μM, between about 50 μM and about 100 μM, between about 50 μM and about 75 μM, between about 75 μM and about 375 μM, between about 100 μM and about 350 μM, between about 125 μM and about 325 μM, between about 150 μM and about 300 μM, between about 175 μM and about 275 μM, or between about 200 μM and about 250 μM. In some embodiments of the method of perfusion MRI, the metal chelate is dosed at a concentration of about 50 μM, about 75 μM, about 100 μM, about 125 μM, about 150 μM, about 175 μM, about 200 μM, about 225 μM, about 250 μM, about 275 μM, about 300 μM, about 325 μM, about 350 μM, about 375 μM, about 400 μM, or any range between any two of these values, including endpoints. In some embodiments of the method of perfusion MRI, the metal chelate is dosed at a concentration between about 50 μM and about 400 μM.

In some embodiments of the method of perfusion MRI, the metal chelate is dosed at a concentration between about 0.05 mmol/kg body wt to about 1.0 mmol/kg body wt, about 0.1 mmol/kg body wt to about 1.0 mmol/kg body wt, about 0.2 mmol/kg body wt to about 1.0 mmol/kg body wt, about 0.3 mmol/kg body wt to about 1.0 mmol/kg body wt, about 0.4 mmol/kg body wt to about 1.0 mmol/kg body wt, about 0.5 mmol/kg body wt to about 1.0 mmol/kg body wt, about 0.6 mmol/kg body wt to about 1.0 mmol/kg body wt, about 0.7 mmol/kg body wt to about 1.0 mmol/kg body wt, about 0.8 mmol/kg body wt to about 1.0 mmol/kg body wt, about 0.9 mmol/kg body wt to about 1.0 mmol/kg body wt, about 0.05 mmol/kg body wt to about 0.9 mmol/kg body wt, about 0.05 mmol/kg body wt to about 0.8 mmol/kg body wt, about 0.05 mmol/kg body wt to about 0.7 mmol/kg body wt, about 0.05 mmol/kg body wt to about 0.6 mmol/kg body wt, about 0.05 mmol/kg body wt to about 0.5 mmol/kg body wt, about 0.05 mmol/kg body wt to about 0.4 mmol/kg body wt, about 0.05 mmol/kg body wt to about 0.3 mmol/kg body wt, about 0.05 mmol/kg body wt to about 0.2 mmol/kg body wt, about 0.05 mmol/kg body wt to about 0.1 mmol/kg body wt. about 0.1 mmol/kg body wt to about 0.9 mmol/kg body wt, about 0.2 mmol/kg body wt to about 0.8 mmol/kg body wt, about 0.3 mmol/kg body wt to about 0.7 mmol/kg body wt, or about 0.4 mmol/kg body wt to about 0.6 mmol/kg body wt. In some embodiments of the method of perfusion MRI, the metal chelate is dosed at a concentration of about 0.05 mmol/kg body wt, about 0.1 mmol/kg body wt, about 0.2 mmol/kg body wt, about 0.3 mmol/kg body wt, about 0.4 mmol/kg body wt, about 0.5 mmol/kg body wt, about 0.6 mmol/kg body wt, about 0.7 mmol/kg body wt, about 0.8 mmol/kg body wt, about 0.9 mmol/kg body wt, about 1.0 mmol/kg body wt, or any range between any two of these values, including endpoints. In some embodiments of the method of perfusion MRI, the metal chelate is dosed at a concentration between about 0.05 mmol/kg body wt to about 1.0 mmol/kg body wt. In an embodiment of the method of perfusion MRI, the metal chelate is dosed at a concentration of about 0.2 mmol/kg body wt.

In some embodiments of the method of perfusion MRI, the region of the tissue is hypoxic, as described herein.

In another aspect, the present invention provides a method of detecting, monitoring, and diagnosing hypoxia, the method comprising applying the metal chelate of the present invention to a tissue, detecting the metal chelate in a region of the tissue using MRI, comparing the level of detected metal chelate to a comparator, and determining that the tissue is hypoxic when the level of metal chelate is higher compared to a comparator.

In some embodiments, the metal of the metal chelate is any metal disclosed herein. For example, in some embodiments, the metal of the metal chelate is selected from iron, copper, cobalt, manganese, nickel, zinc, zirconium, gallium, neodymium, europium, gadolinium, terbium, dysprosium, holmium, erbium, thulium, ytterbium, lutetium, or any combination thereof. In some embodiments, the metal of the metal chelate is iron.

In some embodiments, the iron has an oxidation state of +2 or +3. In some embodiments, the iron has an oxidation state of +3.

In some embodiments, the metal chelate is dosed at a concentration up to about 400 μM. In some embodiments, the metal chelate is dosed at a concentration between about 50 μM and about 400 μM, between about 75 μM and about 400 μM, between about 100 μM and about 400 μM, between about 125 μM and about 400 μM, between about 150 μM and about 400 μM, between about 175 μM and about 400 μM, between about 200 μM and about 400 μM, between about 225 μM and about 400 μM, between about 250 μM and about 400 μM, between about 275 μM and about 400 μM, between about 300 μM and about 400 μM, between about 325 μM and about 400 μM, between about 350 μM and about 400 μM, between about 375 μM and about 400 μM, between about 50 μM and about 375 μM, between about 50 μM and about 350 μM, between about 50 μM and about 325 μM, between about 50 μM and about 300 μM, between about 50 μM and about 275 μM, between about 50 μM and about 250 μM, between about 50 μM and about 225 μM, between about 50 μM and about 200 μM, between about 50 μM and about 175 μM, between about 50 μM and about 150 μM, between about 50 μM and about 125 μM, between about 50 μM and about 100 μM, between about 50 μM and about 75 μM, between about 75 μM and about 375 μM, between about 100 μM and about 350 μM, between about 125 μM and about 325 μM, between about 150 μM and about 300 μM, between about 175 μM and about 275 μM, or between about 200 μM and about 250 μM. In some embodiments, the metal chelate is dosed at a concentration of about 50 μM, about 75 μM, about 100 μM, about 125 μM, about 150 μM, about 175 μM, about 200 μM, about 225 μM, about 250 μM, about 275 μM, about 300 μM, about 325 μM, about 350 μM, about 375 μM, about 400 μM, or any range between any two of these values, including endpoints. In some embodiments, the metal chelate is dosed at a concentration between about 50 μM and about 400 μM.

In some embodiments, the metal chelate is dosed at a concentration between about 0.05 mmol/kg body wt to about 1.0 mmol/kg body wt, about 0.1 mmol/kg body wt to about 1.0 mmol/kg body wt, about 0.2 mmol/kg body wt to about 1.0 mmol/kg body wt, about 0.3 mmol/kg body wt to about 1.0 mmol/kg body wt, about 0.4 mmol/kg body wt to about 1.0 mmol/kg body wt, about 0.5 mmol/kg body wt to about 1.0 mmol/kg body wt, about 0.6 mmol/kg body wt to about 1.0 mmol/kg body wt, about 0.7 mmol/kg body wt to about 1.0 mmol/kg body wt, about 0.8 mmol/kg body wt to about 1.0 mmol/kg body wt, about 0.9 mmol/kg body wt to about 1.0 mmol/kg body wt, about 0.05 mmol/kg body wt to about 0.9 mmol/kg body wt, about 0.05 mmol/kg body wt to about 0.8 mmol/kg body wt, about 0.05 mmol/kg body wt to about 0.7 mmol/kg body wt, about 0.05 mmol/kg body wt to about 0.6 mmol/kg body wt, about 0.05 mmol/kg body wt to about 0.5 mmol/kg body wt, about 0.05 mmol/kg body wt to about 0.4 mmol/kg body wt, about 0.05 mmol/kg body wt to about 0.3 mmol/kg body wt, about 0.05 mmol/kg body wt to about 0.2 mmol/kg body wt, about 0.05 mmol/kg body wt to about 0.1 mmol/kg body wt. about 0.1 mmol/kg body wt to about 0.9 mmol/kg body wt, about 0.2 mmol/kg body wt to about 0.8 mmol/kg body wt, about 0.3 mmol/kg body wt to about 0.7 mmol/kg body wt, or about 0.4 mmol/kg body wt to about 0.6 mmol/kg body wt. In some embodiments, the metal chelate is dosed at a concentration of about 0.05 mmol/kg body wt, about 0.1 mmol/kg body wt, about 0.2 mmol/kg body wt, about 0.3 mmol/kg body wt, about 0.4 mmol/kg body wt, about 0.5 mmol/kg body wt, about 0.6 mmol/kg body wt, about 0.7 mmol/kg body wt, about 0.8 mmol/kg body wt, about 0.9 mmol/kg body wt, about 1.0 mmol/kg body wt, or any range between any two of these values, including endpoints. In some embodiments, the metal chelate is dosed at a concentration between about 0.05 mmol/kg body wt to about 1.0 mmol/kg body wt. In an embodiment of the method of perfusion MRI, the metal chelate is dosed at a concentration of about 0.2 mmol/kg body wt.

In various embodiments of the methods of the invention, the level of detected metal chelate is determined to be increased when the level of detected metal chelate is increased by at least 0.1%, by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared to a comparator.

In various embodiments of the methods of the invention, the level of detected metal chelate is determined to be increased when the level of detected metal chelate is increased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared to a comparator.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1—Synthesis of Formula (I)

DFOB mesylate, HBTU, HOBT, DIPEA and reversed phase silica were purchased from Sigma-Aldrich (USA). TFA, DMF, Methylene chloride, Methanol, Acetonitrile were purchased from Oakwood Products, Inc. (USA) and used without further purification. Unless otherwise mentioned, the water used for all the experiments requiring water was ultrapure (Millipore) water. The dialysis tubes with a molecular weight cut-off of 100-500 Da were purchased from Spectrum Laboratories Inc. (USA). Cell culture media reagents were from Genesee (USA). Alamar blue was obtained from Bio-Rad Laboratories, Inc. (USA).

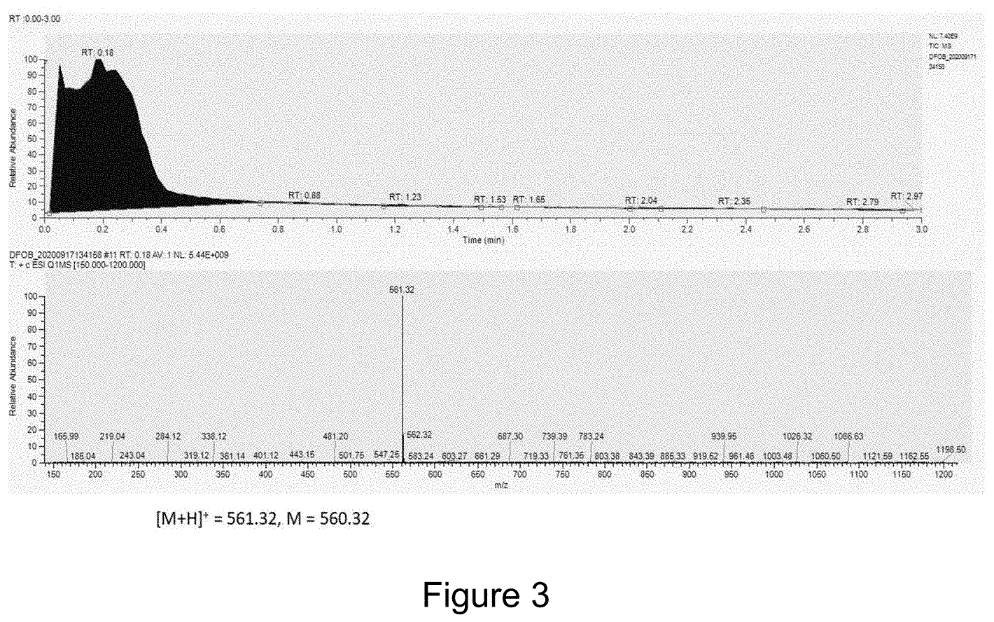

Synthesis of (6-(2-nitro-1H-imidazol-1-yl)hexanoic acid) (INT-02)—2-nitroimidazole 1.05 equivalents of ethyl 6-bromohexanoate and 2.5 equivalents of potassium carbonate were mixed in acetonitrile. The mixture was stirred using a magnetic stirrer under reflux condition for one week. Afterwards, the mixture was cooled down to room temperature and filtered using gravity filtration and the solvent was removed under reduced pressure. The residual then was dissolved in ethyl acetate and followed by three times washing with water and drying with sodium sulfate. Finally, the solvent was removed under reduced pressure to yield (77%) the intermediate product, ethyl 6-(2-nitro-1H-imidazol-1-yl) hexanoate. The product was identified using ¹H NMR spectroscopy with chloroform-d as the solvent. INT-02 was obtained by dissolving ethyl 6-(2-nitro-1H-imidazol-1-yl) hexanoate in a minimal amount of concentrated HCl and stirring overnight and removing the solvent under reduced pressure to yield (99%) a yellow oil. INT-02 ¹H NMR (400 MHz, DMSO) δ 7.69 (s, 1H), 7.16 (s, 1H), 4.36 (t, J=7.3 Hz, 2H), 2.20 (t, J=7.3 Hz, 2H), 1.76 (m, 2H), 1.52 (m, 2H), 1.27 (m, 2H). ESI-MS was able to detect the INT-01 at 561.2 (FIG. 3) in positive mode.

INT-03 ¹H NMR (400 MHz, CDCl₃) δ 7.11 (m, 1H), 7.07 (d, J=0.8 Hz, 1H), 4.39 (t, J=7.3 Hz, 2H), 4.09 (q, J=7.1 Hz, 2H), 2.28 (t, J=7.1 Hz, 2H), 1.85 (m, 2H), 1.65 (m, 2H), 1.36 (m, 2H), 1.22 (t, J=0.9 Hz, 3H).

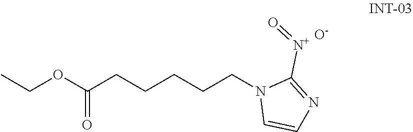

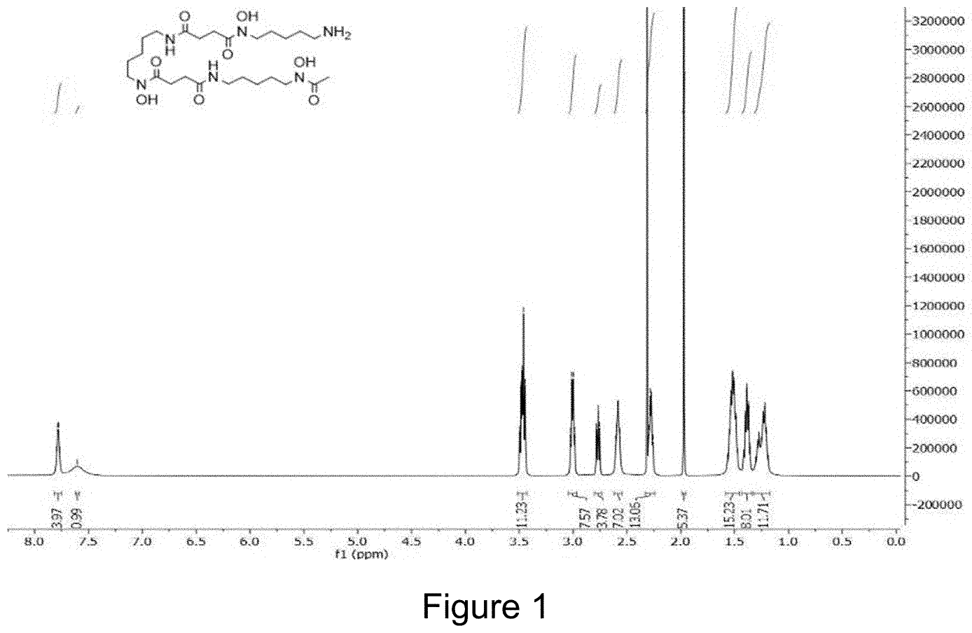

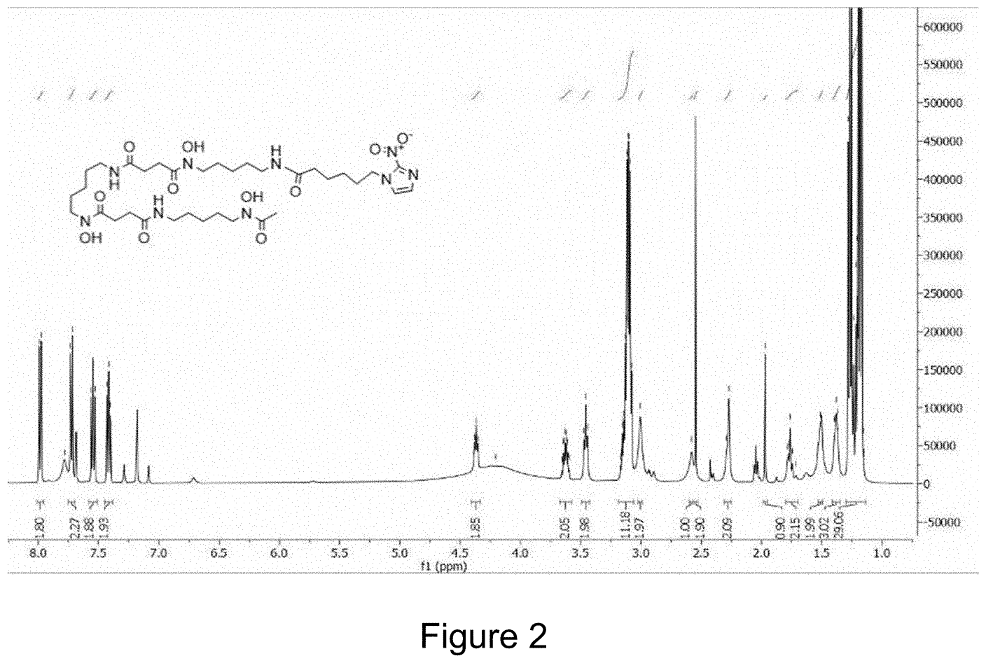

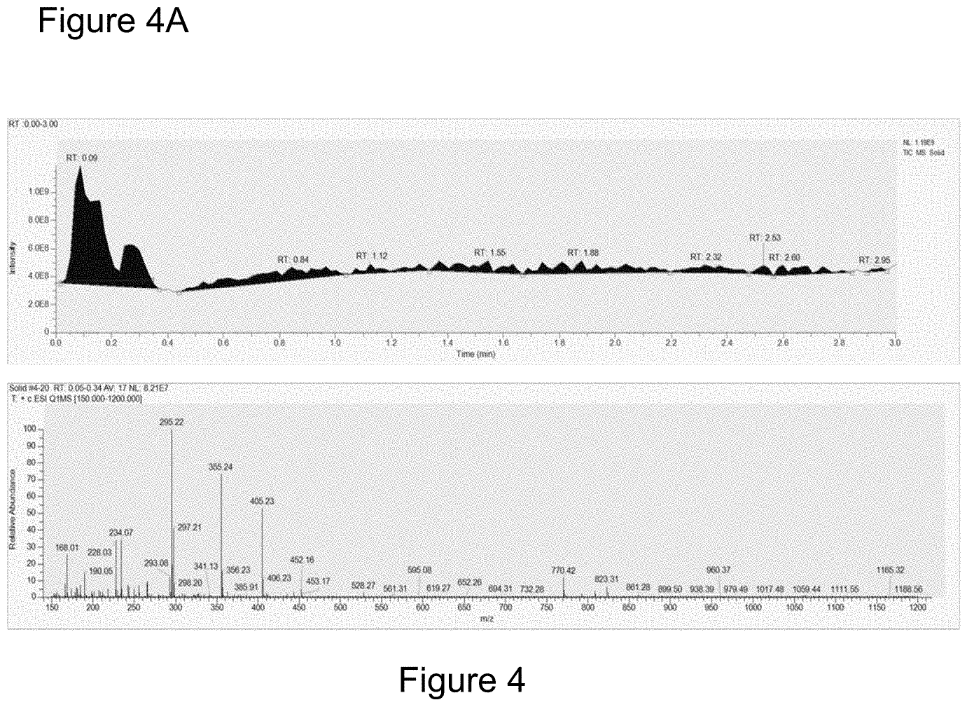

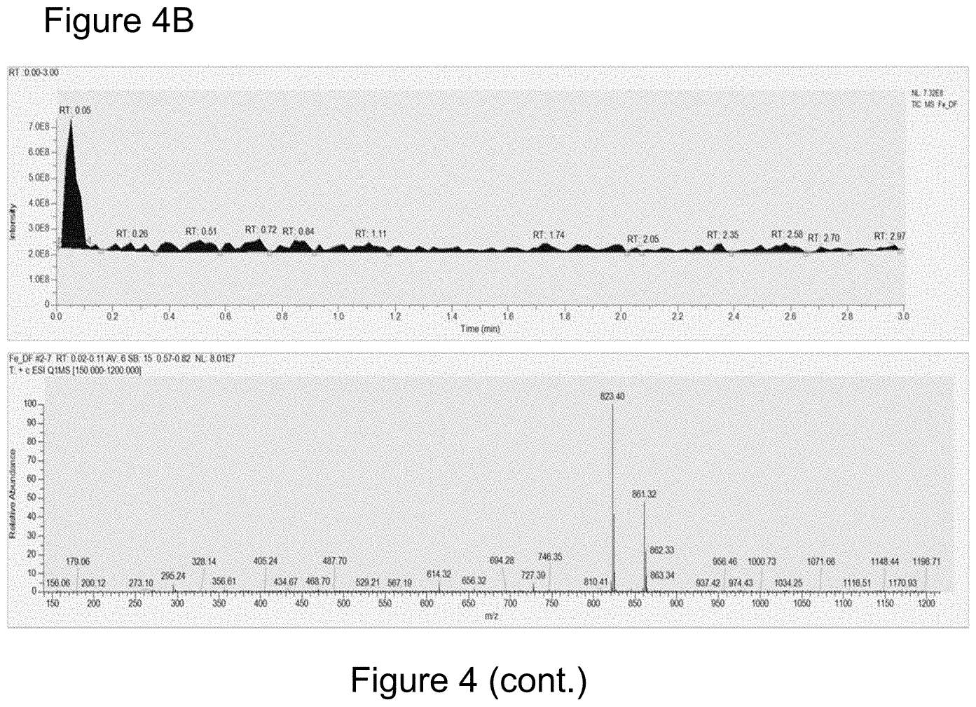

Synthesis of Formula (I)—To a suspension of INT-01 and INT-02 in a ratio of 1:1.2 in DMF was added HBTU, and HOBt in DMF and heated under N2 at 50° C. for 1 h. Then the mixture was cooled down to room temperature followed by addition of DIPEA and stirring the suspension under N2 flow overnight at RT. The reaction was stopped by placing the reactor in ice cold water. Later the solvent was removed under reduced pressure. The residue was suspended in cold acetonitrile by sonication and the precipitate was collected to eliminate HBTU, HOBt, and other side products, if any. This step was repeated three times. Nuclear magnetic resonance (NMR) spectra for characterization of the compound were recorded on two Bruker NMR systems operating at 400 MHz and 500 MHz fields. Electrospray ionization mass spectrometry (ESI-MS) was performed on a Bruker system at Arizona state university. Performing ESI-MS and ¹H NMR on the solid confirmed the presence of DFOBNI. The collected solid from reversed phase silica column chromatography was then dissolved in water and further purified on a reversed phase silica column using a 1-1 ratio of water and methanol as elute system. The solvent was removed using a high vacuum and the solid was used to chelate iron. INT-01 ¹H NMR is shown in FIG. 1 and Formula (I)¹H NMR is shown in FIG. 2. ESI-MS in positive mode and showed a peak at 770 (FIG. 4A and FIG. 4B) that can be attributed to Formula (I).

Formation of the metal chelate—A 1:1 ratio of Formula (I) and FeCl₃ was mixed and the mixture was stirred for 1 h. The pH of the stirred mixture was kept in acidic to neutral conditions to reduce the possibility of the formation of insoluble ferric ion in an aqueous medium. Then the mixture was poured in a dialysis tube with a molecular weight cut-off of 500 Da and placed in water to remove the unchelated iron; the dialysis process was repeated three times to ensure the mixture has no free iron.

The iron atom has a molar mass of about 56 g/mol, and when it is chelated by Formula (I) to generate metal chelate three hydrogens leave Formula (I) resulting in 823 molecular mass for the metal chelate. Free iron ions can induce cytotoxicity and also influence paramagnetic properties. To eliminate the free iron ions from the product and small molecule impurities dialysis tubes with a molecular weight cut off of 500 Da were used. Dialysis was performed for an entire period of 48 h in excess water. The water was stirred with a magnet stirrer and replace with fresh water three times to ensure the elimination of the free ions and impurities as shown in FIG. 4B).

### Example 2—Cytotoxicity

To study the cytotoxicity of DFOB, NIH-3T3 fibroblast cells were used. The cells were exposed to different concentrations of DFOB for 4, 8, and 24 h under either hypoxic or normoxic conditions. Afterwards, the cells were washed with PBS and exposed to 10% Alamar blue in media for 4 h in their respective conditions. To assess the viability of the cells 100 l of the exposed Alamar blue was collected in a 96-well plate using the spectrophotometer the absorbance was measured at 570 nm and 600 nm excitations and the viability of the cells was calculated.

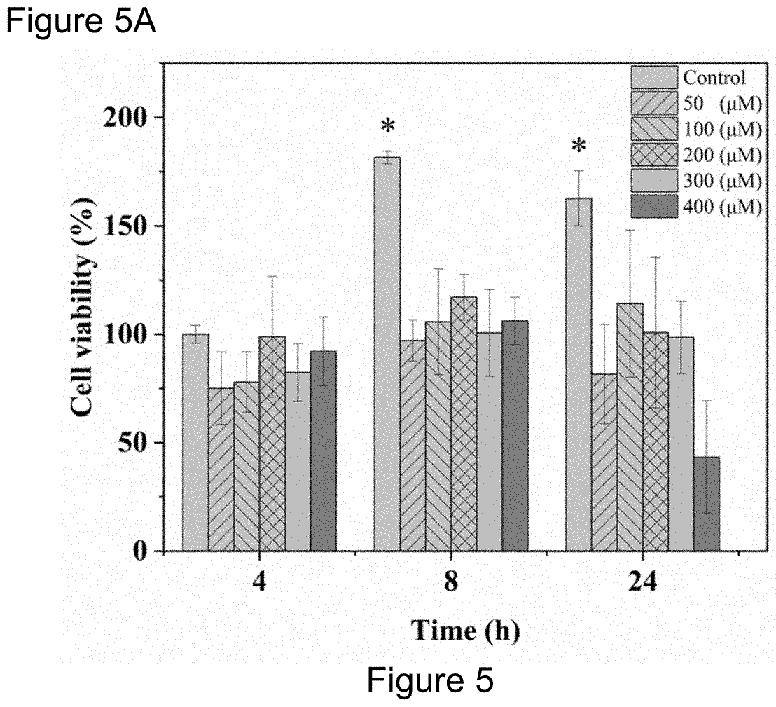

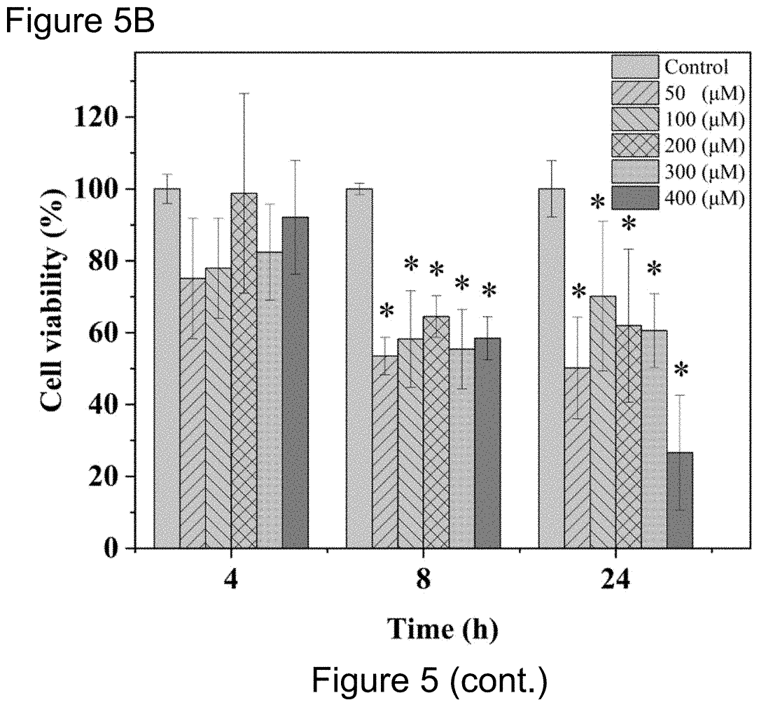

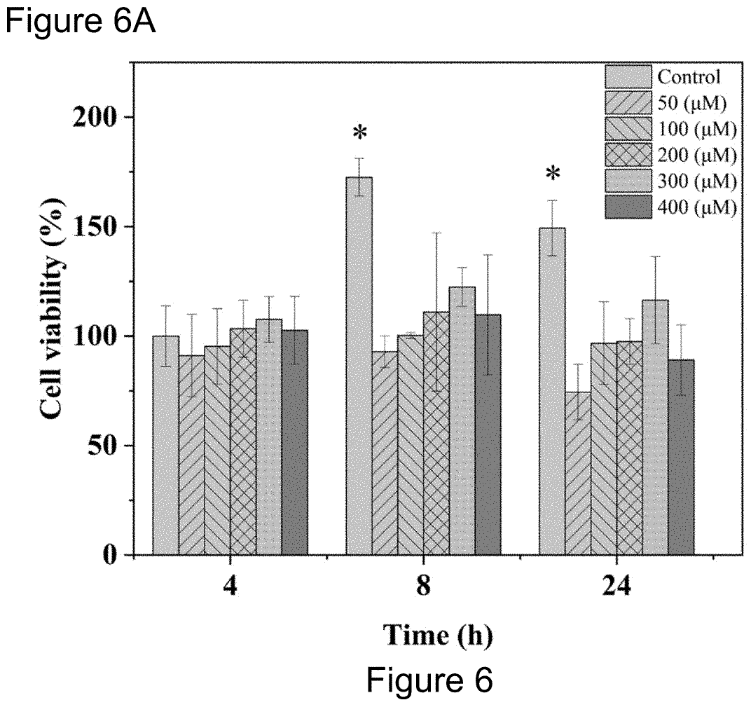

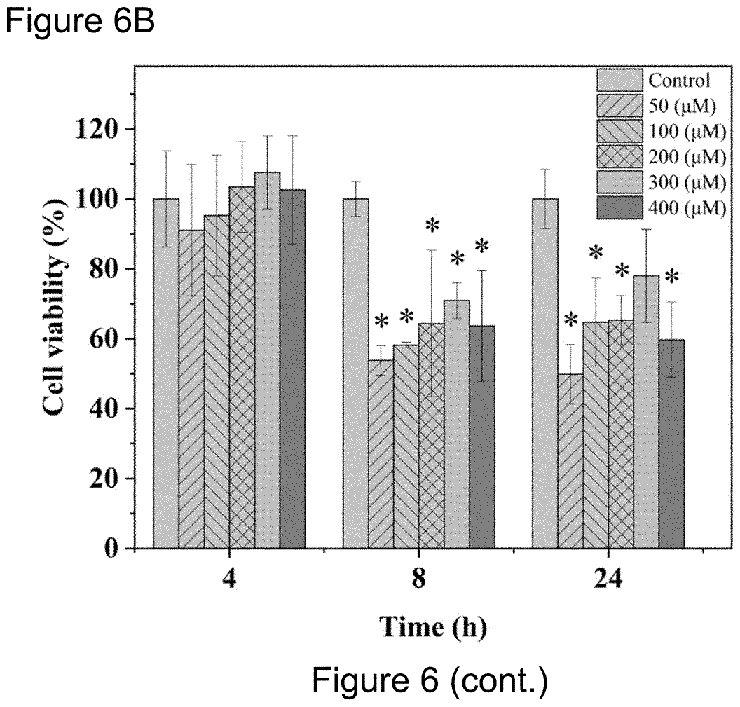

Cytotoxicity assay for FOBNI was a viability test using Alamar blue. The results are summarized in FIG. 5 for normoxic conditions and FIG. 6 for hypoxic conditions. The data showed no significant reduction (n=3) in the viability after up to 24 h exposure of the cells to contrast agent at the concentrations of 100-400 μM in hypoxic and normoxic conditions when the control of the 4 h exposure used to assess the viability. However, when the control of each time point used to assess the viability, a significant drop in cell viability after 8 h and 24 h of exposure in the fibroblast cells in hypoxic and normoxic conditions was noticed. The increased viability, which is a translation of reduced Alamar blue in control samples of 8 h and 24 h can be attributed to the increased number of the cells in the control wells. After seeding the cells the cell number in the control wells could increase and that can influence the amount of reduced compound in absorbance test.

The cell viability assay confirmed the cytocompatibility of the chelated DFOBNI up to 8 h or direct exposure to the cells. The results are supported by previous studies characterizing similar derivatives of FOB for different purposes. The body's immediate clearance response for the injected molecules eliminates the contrast agent from the bloodstream culminating in no injected molecules in proximity to the normal cells for an extended period. This is ascribed to the functional blood circulatory system, eliminating the perfusion and diffusion-limited hypoxic regions in normal and healthy organs. It is reported the circulation half-time for DFOB is about 5 min in mouse. On the other hand, the prolonged exposure of the contrast agent affected the cells' viability, which could be considered the auxiliary effect of the molecule. It can mainly provide spatial distribution maps of hypoxia in MR modality and serve as a toxic molecule for hypoxic cells due to the covalent bond of the nitroimidazole moiety and the thiol proteins and the continued exposure of the cells to the contrast agent. The cytotoxicity assay under normoxic and hypoxic conditions did not show any significant difference in the final result this could indicate the toxicity is just resulting from prolonged exposure to the contrast agent. The retention would happen when the contrast is chemically bond to the hypoxic regions in the organs.

### Example 3—Relaxometry

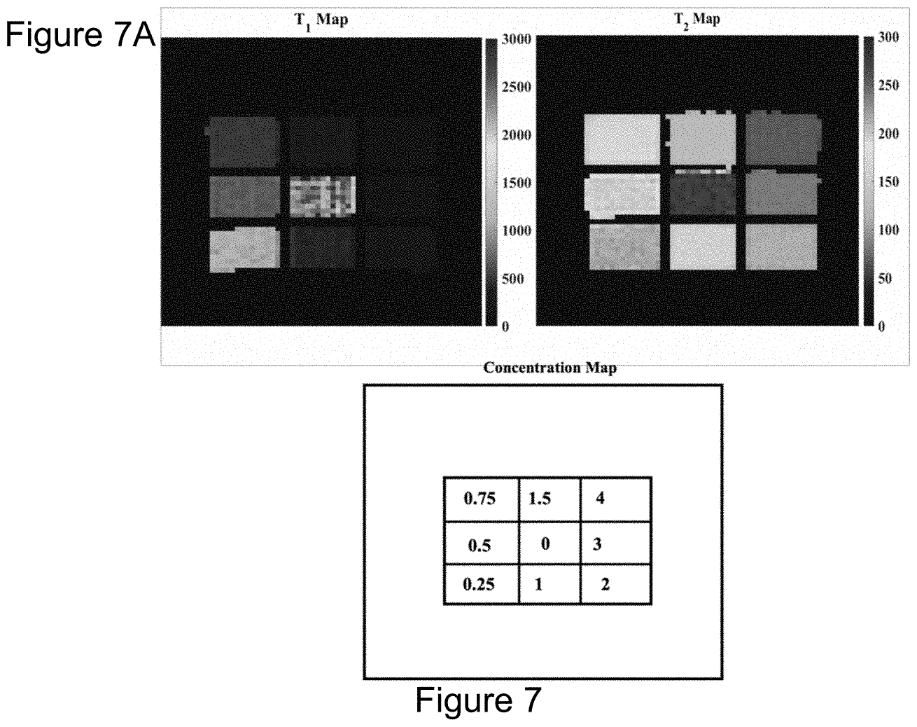

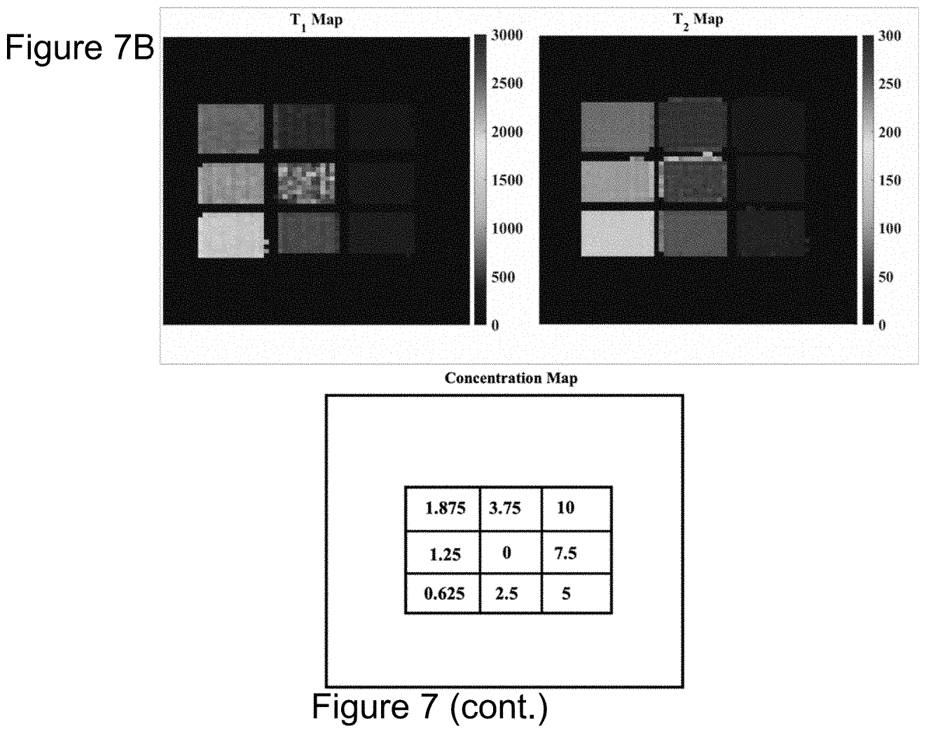

The basic paramagnetic properties i.e., relaxivity of chelated DFOB (FOB) and FOBNI was measured at 7T using a preclinical Bruker MRI scanner. The relaxivity of the two molecules were studied using a home-made 3D printed phantom capable of controlling temperature. The phantoms contained nine different concentrations of each contrast agents in water from 0-4 mM for FOB and 0-10 mM for FOBNI. The imaging parameters for this study were as matrix size 64×64, the field of view 2.56 cm×2.56 cm and TR=205-5000 ms, TE=10 ms for the T₁ map and TR=5000 ms, TE=8-200 ms for the T₂ map. The MR images were later analyzed in a homemade MATLAB code to calculate the T₁ and T₂ values for each pixel. Using the T₁ and T₂ values for each concentration and Rᵢ=[C]rᵢ+Rᵢ⁰ equation the relaxivity values were calculated by a linear fit on Rᵢ and [C]. Where Rᵢ (i=1 or 2) is the relaxation rate of the tissue, [C] is the concentration of the contrast agent, ri is the relaxivity of the contrast agent and the Rᵢ⁰ is the intrinsic relaxation rate of the water. FIG. 7A and FIG. 7B show the T₁ and T₂ maps for the phantom and a concentration map indicating the concentrations of FOB and FOBNI in each respective well.

Considering the Solomon-Bloembergen-Morgan (SBM) theory one of the factors that affects the r₁ value of a chelating molecule is the number of inner sphere water molecules known as hydration (q). In a successful conjugation reaction it is assumed the primary amine group transforms to an amide group. The addition of the nitroimidazole moiety can influence the hydration of FOBNI, which is the dominant mechanism in determining the longitudinal relaxivity of a contrast agent. The behavior of r₁ and r₂ in the direction of changes is usually the same meaning with the increase of the r₁ the r₂ also increases, or the decrease in r₁ is also followed by a decrease in r₂ values. This follows the simplified SBM equation that takes into account the hydration number. However, there are reports that do not follow this simplistic approach and require more rigorous analysis and simulation. FOBNI shows about 75% decrease in r₁ value compare to FOB and about 65% increase in r₂ value. Generally, the MR contrast agents alter both T₁ and T₂ relaxation times. The extent to which a contrast agent is categorized as T₁ or T₂ (positive contrast or negative contrast) lies in the ratio of its transverse to longitudinal relaxivity. The ratio r₂/r₁>>1 indicate a T₂ contrast agent while r₂/r₁<1 can imply a T₁ contrast agent. However, as some endogenous factors and imaging artifacts result in negative contrast as well, the positive contrast is favorable in MR images and if a contrast agent is capable of producing strong T₁ contrast the discussion of the relaxivity ratio is overlooked. For example, GdDO3NI with a relaxivity ratio of 1.58 would generate a better contrast in T₂ images however with a longitudinal relaxivity r₁ of 4.75 s⁻¹ mmol⁻¹L it can furnish a strong positive contrast. The relaxivity ratio of 8.84 for FOBNI with longitudinal relaxivity of 0.51 s⁻¹ mmol⁻¹ L, which emphasizes its high performance to generate hypointense regions in T₂ weighted images.

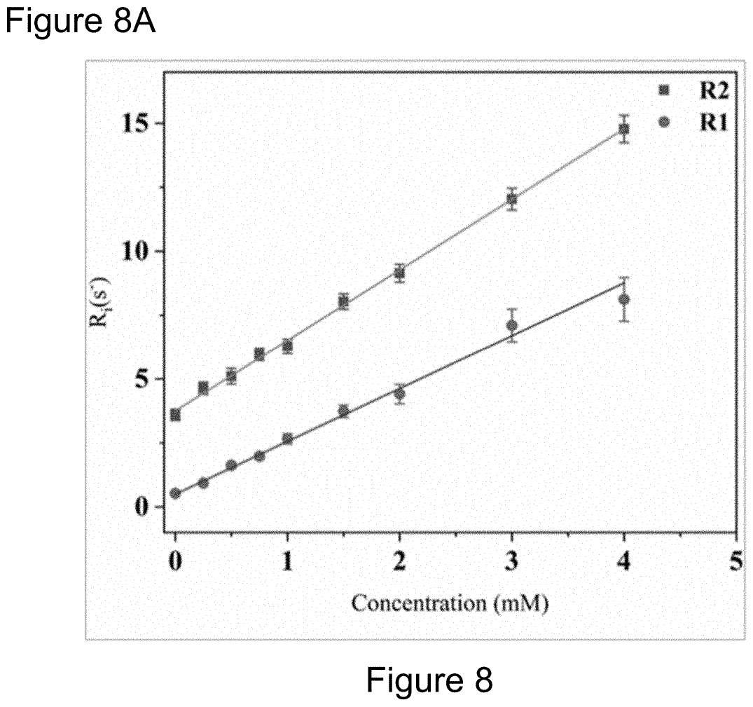

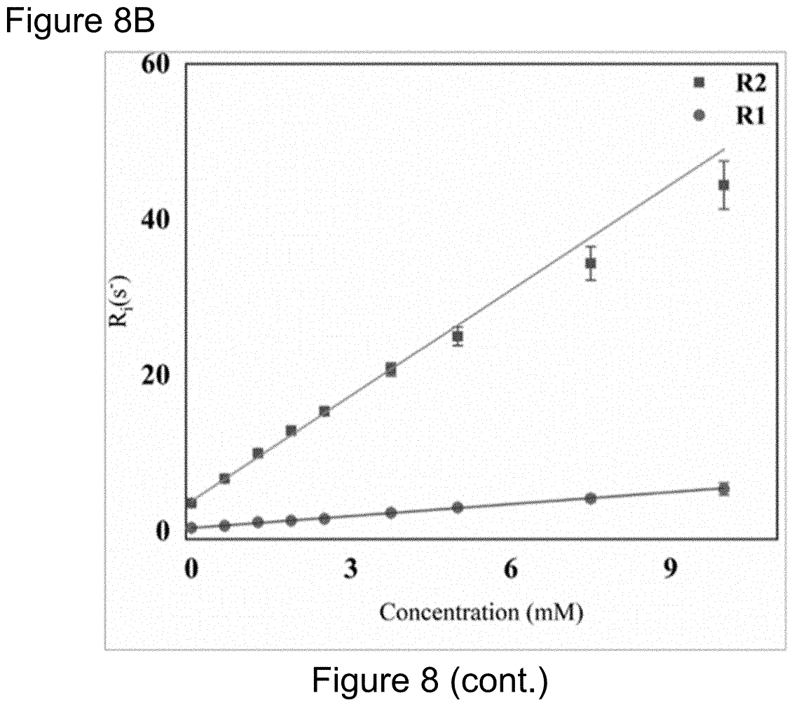

FIG. 8 represents the result of the linear fit for R₁ and R₂ for the two contrast agents. The fits assessed by the r² values and all the four data sets showed r²>0.99. Using the linear fit function, the following equations are acquired for relaxation rate vs concentration, R₁=(2.06±0.06)[C]+(0.49±0.04) (in red) and R₂=(2.75±0.06)[C]+(3.75±0.08) (in gray) for FOB and R₁=(0.51±0.1)[C]+(0.44±0.03) (in red) and R₂=(4.51±0.14)[C]+(3.87±0.19) (in gray) for FOBNI.

### Example 4—In Vitro Studies

The in vitro study for hypoxia-targeting efficiency of FOBNI was assessed in a hypoxia chamber glove box capable of adjusting oxygen concentration, temperature and CO₂ and equipped with a small centrifuge unit. The 3T₃ fibroblast cells were grown in a T-150 flask once they reached the confluency of 80% divided equally to five T-75 flasks to ensure the flasks were identical before being exposed to contrast agents and different conditions. The cells were supplied with Dulbecco's Modified Eagle Medium (DMEM), fetal bovine serum (10%), and penicillin/streptomycin (1%). Once the five flasks reached the confluency of 75-80% the growth media were replaced with the following mediums.

Two flasks were supplied with fresh culture media supplemented with DFOB, two with fresh media and FOBNI and one with just fresh media as control. The control and one FOB and one FOBNI labeled flasks were placed in a regular cell incubator at 21% O₂, 5% CO₂ and 37° C. and the remaining two flasks were transferred to the hypoxia chamber at 1% O₂, 5% CO₂ and 37° C. All the samples were kept at defined conditions for 4 h before removing the media. The cells were washed three times with phosphate buffered saline (PBS) at the end of four hour incubation and were detached by being exposed to trypsin for 5 min. The collected cell mixtures in Eppendorf Tube were centrifuged at 1000 rpm for 5 min and the medium was removed. Afterwards, the cell pellets were suspended in PBS and again centrifuged three times to ensure the removal of unbound or free DFOB or FOBNI. Finally, to keep the cell pellets intact the tubes were filled with low temperature liquid agar and placed in the fridge to solidify.

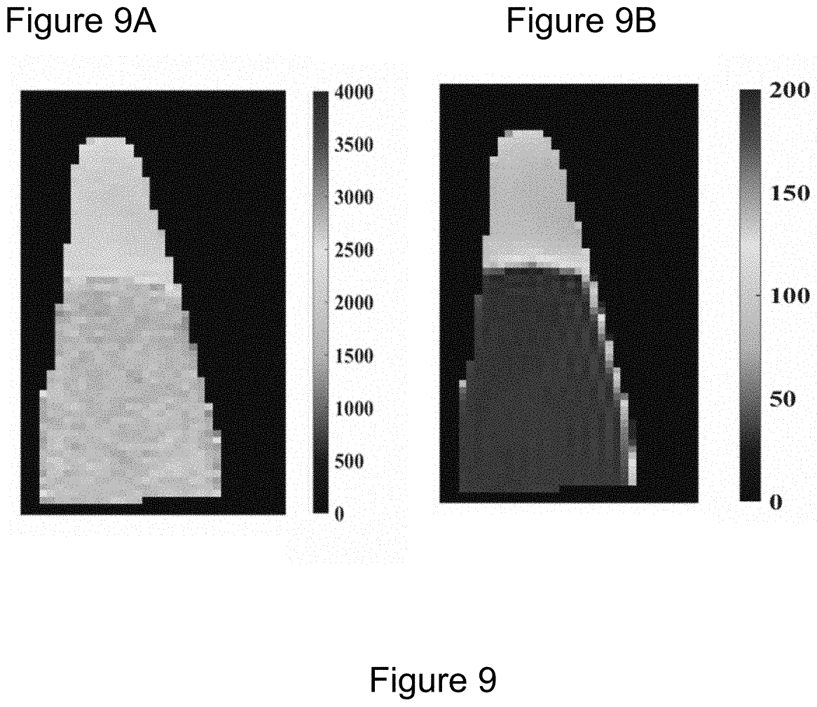

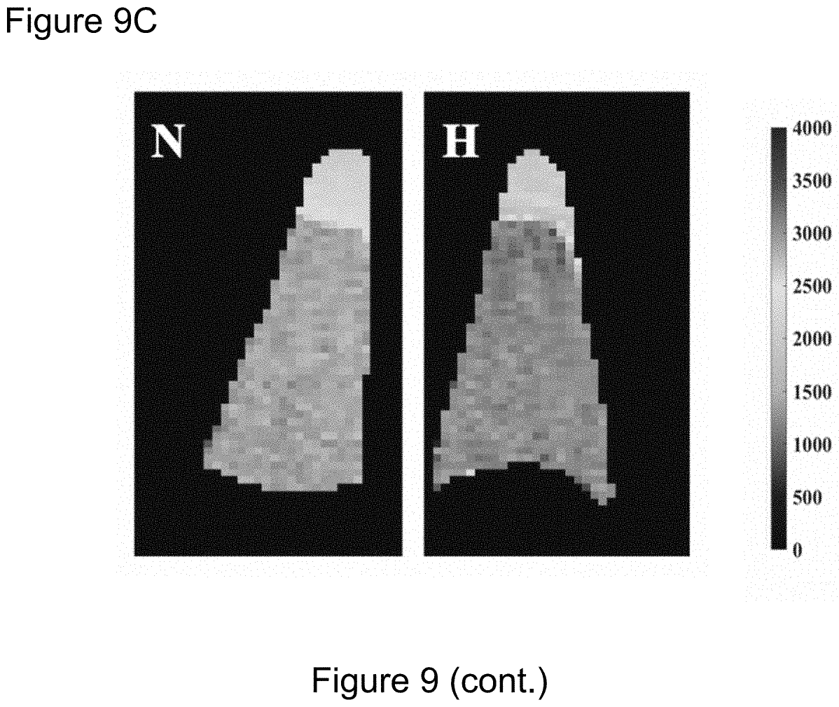

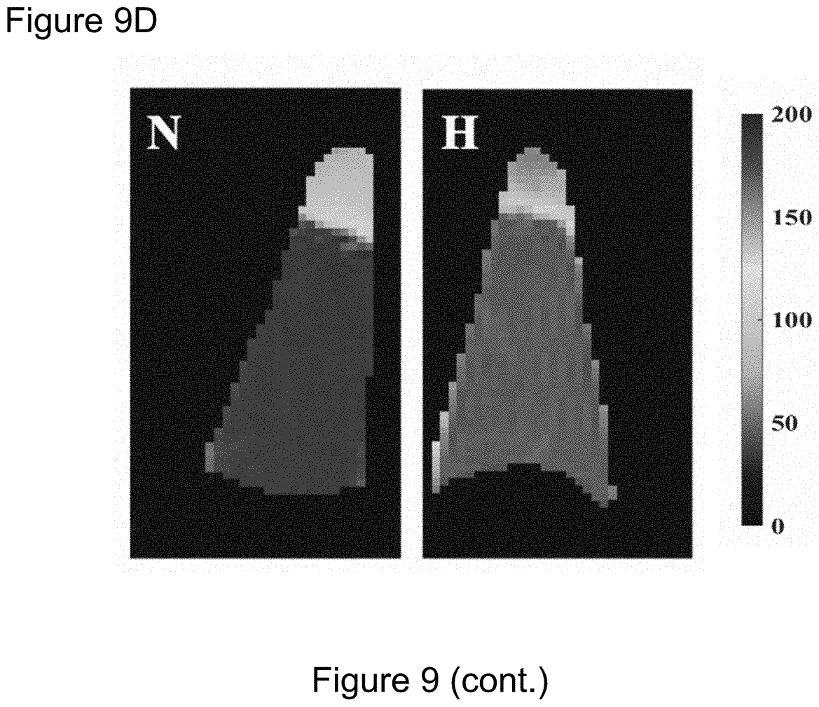

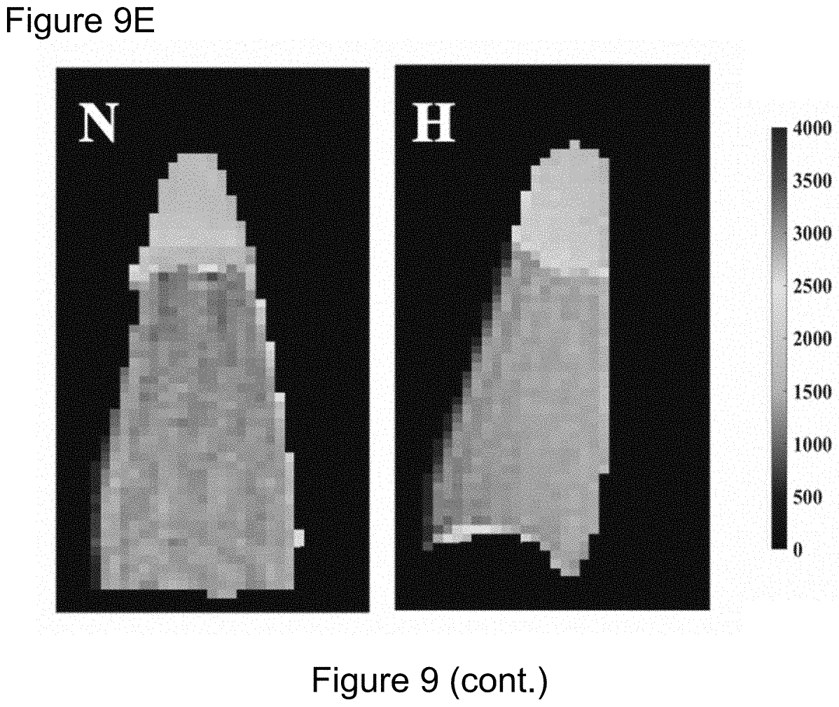

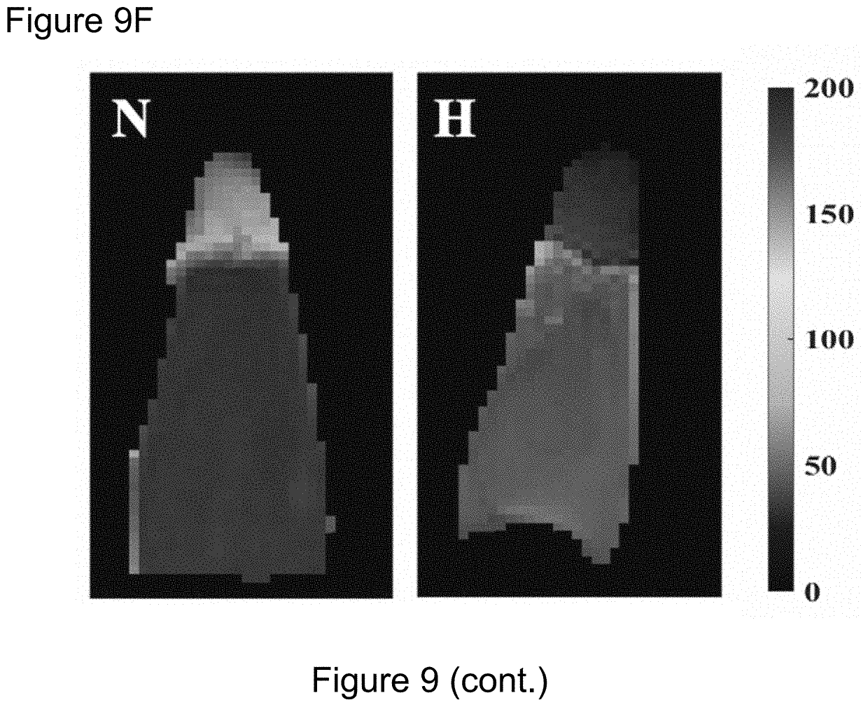

MR Imaging—The samples were imaged using a rat brain surface coil at 7 T. The voxel wise T₁ and T₂ maps of the samples were calculated using MATLAB based software and the images from Variable Repetition Time Fast Spin Echo (VTR) Sequence and multi-echo spin echo sequence, respectively. The VTR sequence was performed with TE=8 ms and thirteen TR values ranging between (205-5000 ms). The images in multi-echo sequence were acquired with TE ranging between 8-200 ms and TR=5000 ms. The field of view was 2.56 cm×2.56 cm and the matrix size of 64×64 for all the experiments. The ROI analysis to calculate the T₁ and T₂ values for cell pellets were performed in MATLAB. FIG. 9 shows the result of MRI for the in vitro experiment. The quantitative values are summarized in Table 1.

TABLE 1
The relaxation times calculated for the control cells, FOB exposed cells under normoxic
(N) and hypoxic (H) conditions and FOBNI exposed cells under normoxic (N) and hypoxic (H)
conditions. The (*) shows statistical significance compared to the control.

| Relaxation | | | | | |
|---|---|---|---|---|---|
| times (ms) | Control | FOB (N) | FOB (H) | FOBNI (N) | FOBNI (H) |
| T₁ | 2098 ± 106 | 2280 ± 222(*) | 2002 ± 143(*) | 1793 ± 165(*) | 1909 ± 162(*) |
| T₂ | 90.2 ± 12.6 | 89.9 ± 11.21 | 75.5 ± 15.0(*) | 59.3 ± 9.3(*) | 32.4 ± 3.4(*) |

Statistics—All results are reported as means±standard deviations (SD). For statistical comparisons unpaired t-test was employed and confidence intervals α=0.05, 95% was used between specific means.

The control cells showed a T₁ value of 2098±106 ms and T₂ value of 90.2±12.6 ms. The T₁ and T₂ values cells exposed to FOB under normoxic conditions were 2280±222 ms and 89.9±11.21 ms and under hypoxic conditions were 2002±143 ms and 75.5±15.0 ms respectively. However, normoxic values of the cells exposed to FOBNI for T₁ and T₂ were 1793±165 ms and 59.3±9.3 ms respectively, and under hypoxic conditions those were 1909±162 ms and 32.4±3.4 ms. The statistical analysis and unpaired t-test on the volumetric data, i.e. taking into account the entire cell pellet (not just one slice), showed statistically significant changes in T₁ and T₂ values of the control cell sample compared to the exposed cells.

DFOB has very low cell permeability; one approach to improve its permeability is modifying the molecule by adding chemical groups to the molecule that enhances the cellular uptake. Conjugation of DFOB and imidazole significantly improved the cell permeability compare to DFOB. Our results also showed the addition of nitroimidazole moiety to DFOB increased the cellular uptake of FOBNI, which manifested itself in the T₂ values of the exposed cells to FOB (T₂=89.9±11.2 ms) and FOBNI (T₂=59.3±9.3 ms) under normoxic conditions. Using the relaxivity equation for transverse relaxation rate the pellet of normoxic FOB exposed cells shows contrast agent concentration of 2.67 mM and FOBNI exposed cells show 2.88 mM concentration of contrast, which lends credence to increased cell permeability of the contrast agent after the addition of nitroimidazole group. The most significant change in concentration of iron was obtained when cells experienced hypoxic conditions. The concentration for cells under the hypoxic conditions, which were exposed to FOB was 3.45 mM and for the cells exposed to FOBNI was 5.98 mM showing 1.73 fold increase under hypoxic condition. This increased uptake can be ascribed to improvement in cell permeability after addition of nitroimidazole moiety and more importantly can be an indication of the successful reduction and binding of nitroimidazole moiety in FOBNI to the thiol groups under hypoxic condition.

In conclusion, the studies decribed herein provided a synthesis and characterization of novel iron-based hypoxia targeting contrast agent, to eliminate Gd based complications and provide a cheaper and more economical alternative contrast agent to detect hypoxia. More specifically, the present studies successfully synthesized an iron based hypoxia targeting MRI contrast agent designated as “DFOBNI”, which was a conjugate of Desferrioxamine B (DFOB, a clinically approved drug for the treatment of chronic iron overload), that allowed non-invasive detection of hypoxia with MRI. Further, the agent of the present invention allowed the imaging of hypoxic regions that could result from tumor, stroke, injury by MRI as well as enabling perfusion MRI.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A compound having the structure of Formula (I):

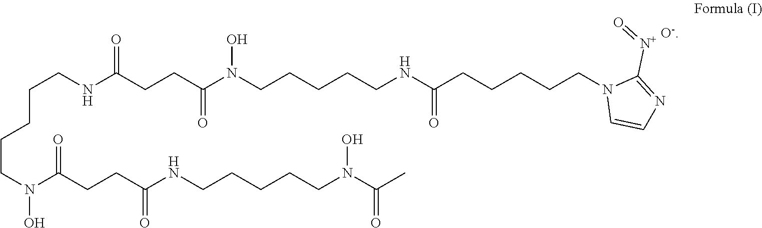

2. A metal chelate comprising a metal and the compound of claim 1.

3. The metal chelate of claim 2, wherein the metal is selected from the group consisting of iron, copper, cobalt, manganese, nickel, zinc, zirconium, gallium, neodymium, europium, gadolinium, terbium, dysprosium, holmium, erbium, thulium, ytterbium, lutetium, and any combination thereof.

4. The metal chelate of claim 2, wherein the metal has an oxidation state of +2 or +3.

5. A process for the preparation of the metal chelate of claim 2, comprising contacting a compound of Formula (I) with a metal complex to form the metal chelate.

6. The process according to claim 5, wherein the metal complex is FeCl₃.

7. The process according to claim 5, further comprising contacting the compound 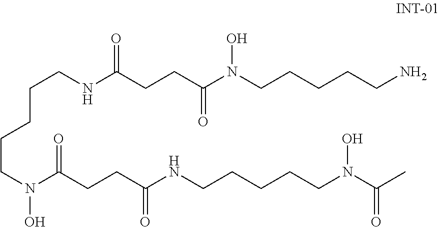 [SMILES: CC(=O)N(O)CCCCCNC(=O)CCC(=O)N(O)CCCCCNC(=O)CCC(=O)N(O)CCCCCN]
with the compound 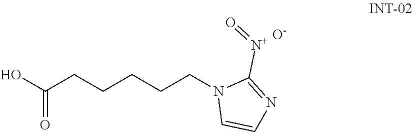
in the presence of a coupling reagent, an amine base, 1-hydroxybenzotriazole monohydrate (HOBt.xH₂O), and a solvent to obtain the compound of Formula (I).

8. The process according to claim 7, wherein the coupling reagent is selected from the group consisting of 3-[bis(dimethylamino)methyliumyl]-3H-benzotriazol-1-oxide hexafluorophosphate (HBTU), benzotriazol-1-yloxytripyrrolidinophosphonium hexafluorophosphate (PyBOP), 1-ethyl-3-(3-dimethylaminopropyl)carbodiimide (EDC), N-hydroxysuccinimide (NHS), and any combination thereof.

9. The process according to claim 7, wherein the amine base is N,N-diisopropylethylamine.

10. The process according to claim 7, further comprising contacting the compound 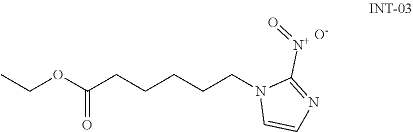
with an acid and a solvent to form the compound (6-(2-nitro-1H-imidazol-1-yl)hexanoic acid) (INT-02).

11. The process according to claim 10, wherein the acid is in a concentration of about 12 M.

12. The process according to claim 10, wherein the acid is HCl.

13. The process according to claim 10, wherein the solvent is selected from the group consisting of water, dimethylformamide (DMF), methanol, dimethyl sulfoxide (DMSO), and any combination thereof.

14. The process according to claim 10, further comprising contacting the compound 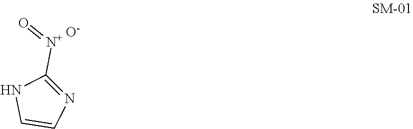
with the compound 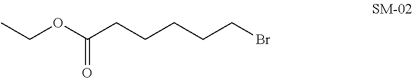
in the in the presence of base and a solvent to form the compound INT-03.

15. The process according to claim 14, wherein the base is selected from the group consisting of K₂CO₃, Na₂CO₃, and any combination thereof.

16. A method of imaging hypoxic tissue regions, the method comprising:
applying the metal chelate of claim 2 to the tissue, and
using magnetic resonance imaging (MRI) to detect the metal chelate.

17. The method of claim 16, wherein the metal chelate is dosed at a concentration up to about 400 μM.

18. The method of claim 16, wherein the metal chelate is dosed at a concentration between about 0.05 mmol/kg body wt to about 1.0 mmol/kg body wt.

19. A method of perfusion magnetic resonance imaging (MRI), the method comprising:
applying the metal chelate of claim 2 to the tissue, and
using MRI to detect the metal chelate in a region of the tissue.

20. The method of claim 19, wherein the region of the tissue is hypoxic.
